(19) United States
(12) Patent Application Publication
(10) Pub. No.: US 2026/0224472 A1
(43) Pub. Date: Aug. 6, 2026

(54) STABLE NON-AQUEOUS GEMCITABINE INJECTION FORMULATION AND PREPARATION METHOD THEREOF

(71) Applicant: Riconpharma LLC, Denville, NJ (US)

(72) Inventors: Preetham Geejula, Hyderabad (IN); SVB Janardhan Garikipati, Hyderabad (IN); Vijaya Kumar Thota, Hyderabad (IN); Praveen Reddy Billa, Denville, NJ (US); Mukteeshwar Gande, Denville, NJ (US)

(73) Assignee: Riconpharma LLC, Denville, NJ (US)

(21) Appl. No.: 19/043,734

(22) Filed: Feb. 3, 2025

(51) Int. Cl. A61K 9/00; A61K 9/08; A61K 31/7068; A61K 47/02; A61K 47/10

(52) U.S. Cl. CPC A61K 9/0019; A61K 9/08; A61K 31/7068; A61K 47/02; A61K 47/10

(57) ABSTRACT

The present invention relates to a stable, non-aqueous Gemcitabine injection formulation. The formulation includes Gemcitabine at a concentration of 100 mg/mL, glycerol at a concentration of 400 mg/mL as a solvent, sodium hydroxide at a concentration of 16 mg/mL as a buffering agent and dehydrated alcohol as a vehicle at a concentration of 50% w/v. The pH of the formulation is adjusted to 6.0 to 10.0 using 1N to 2N alcoholic sodium hydroxide and/or alcoholic hydrochloric acid in the range of 0.1 mg/mL to 1 mg/mL. The formulation can be diluted to 0.1 mg/mL or 10 mg/mL using a normal saline solution comprising 0.9% sodium chloride, maintaining physical and chemical stability for 24 hours at room temperature.

### FIELD OF THE INVENTION

This invention generally relates to pharmaceutical formulations and more specifically, a stable injectable formulation of Gemcitabine for parenteral administration. The invention addresses the challenges associated with stability, solubility, and simplify existing formulations by providing a non-aqueous, glycerol-based composition.

### BACKGROUND OF THE INVENTION

Gemcitabine hydrochloride is one of the most commonly used anticancer drugs. Current Gemcitabine preparations for parenteral administration are predominantly available in the form of lyophilized formulations, such as Gemzar®. While these freeze-dried preparations are widely used, they are associated with significant disadvantages. The preparation process for lyophilized products is complicated and expensive, requiring specialized equipment and conditions. Furthermore, reconstitution before administration adds additional steps, increasing the risk of contamination, medication errors, and occupational hazards for the healthcare personnel involved.

A specific issue with large-volume Gemcitabine solutions was disclosed by Xu et al. (J. Am. Pharm. Assoc., Vol 39, No 4, July/August 1999, pages 509-513). This study reported that when lyophilized Gemcitabine (Gemzar®, Eli Lilly and Company) was reconstituted with aqueous vehicles and stored at 4° C. for 14 days or more, crystals of Gemcitabine formed. These crystals did not dissolve even after warming the solution to room temperature, posing potential risks to the efficacy and safety of the treatment. Commercially available non-aqueous Gemcitabine injection formulations, such as Gemcitabine Injection (Accord Healthcare, Inc.), address some of the challenges of lyophilized formulations but introduce new concerns. These formulations utilize Propylene Glycol and Polyethylene Glycol 300 as solubilizers. The proposed product has reduced number of solubilizers where a single solubilizer Glycerol is used. Glycerol is considered GRAS and is a safe.

### BRIEF SUMMARY OF THE INVENTION

The first aspect of the present invention provides a stable non-aqueous Gemcitabine injection formulation. The formulation includes Gemcitabine at a concentration of 100 mg/mL, glycerol at a concentration of 400 mg/mL as a solvent, sodium hydroxide at a concentration of 16 mg/mL as a buffering agent, and dehydrated alcohol as a vehicle at a concentration of 50% w/v. The formulation is stable at a pH range of 6.0 to 10.0. The pH of the formulation is adjusted using 1N to 2N alcoholic sodium hydroxide and/or alcoholic hydrochloric acid in the range of 0.1 mg/mL to 1 mg/mL.

In an embodiment, the dehydrated alcohol is ethanol.

In another embodiment, the formulation when diluted to concentrations of 0.1 mg/mL and 10 mg/mL with an infusion medium, results in an infusion that remains physically and chemically stable for a period of 24 hours at room temperature.

In yet another embodiment, the infusion medium is a saline solution comprising 0.9% sodium chloride.

The second aspect of the present invention provides a method for preparing a stable non-aqueous Gemcitabine injection formulation. The method includes (i) dissolving Gemcitabine at 60±5° C. in 50% w/v solution of dehydrated alcohol comprising sodium hydroxide at a concentration of 16 mg/mL and glycerol at a concentration of 400 mg/mL to achieve a concentration of 100 mg/mL; (ii) adjusting pH of the solution to 6.0 to 10.0 using 1N to 2N alcoholic sodium hydroxide and/or alcoholic hydrochloric acid in the range of 0.1 mg/mL to 1 mg/mL; and (ii) obtaining the stable non-aqueous Gemcitabine injection formulation after volume adjustment with the dehydrated alcohol.

In an embodiment, the dehydrated alcohol is ethanol.

In another embodiment, method includes preparing a diluted infusion of the formulation at concentrations of 0.1 mg/mL and 10 mg/mL with an infusion medium, which remains physically and chemically stable for 24 hours at room temperature.

In yet another embodiment, the infusion medium is a saline solution comprising 0.9% sodium chloride.

The embodiments covered by this patent are defined by the claims. The summary above provides a general overview of various aspects and introduces some of the concepts that are discussed in greater detail in the following description section. This summary is not meant to identify the key or essential features of the claimed subject matter, nor is it intended to be used on its own to determine the scope of the claims. The subject matter should be understood with reference to the entire specification, including any relevant drawings and the claims themselves.

### BRIEF DESCRIPTION OF THE DRAWINGS

A clear understanding of the key features of the invention summarized above may be had by reference to the appended drawings, which illustrate the method and system of the invention, although it will be understood that such drawings depict preferred embodiments of the invention and, therefore, are not to be considered as limiting its scope with regard to other embodiments which the invention is capable of contemplating. Accordingly:

FIG. 1—FIG. 1 illustrates a method for preparing a stable non-aqueous Gemcitabine injection formulation according to various embodiments of the present invention.

Like reference numerals refer to like parts throughout the several views of the drawings.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion.

Nevertheless, the specification and claims should be read with the understanding that such combinations are entirely within the scope of the invention and the claims. A stable non-aqueous Gemcitabine injection formulation and preparation method thereof is discussed herein. In the following description, for purposes of explanation, numerous specific details are set forth in order to provide a thorough understanding of the present invention. It will be evident, however, to one skilled in the art that the present invention may be practiced without these specific details. The present disclosure is to be considered as an exemplification of the invention and is not intended to limit the invention to the specific embodiments illustrated by the figures or description below. The present invention will now be described by referencing the appended figures representing preferred embodiments.

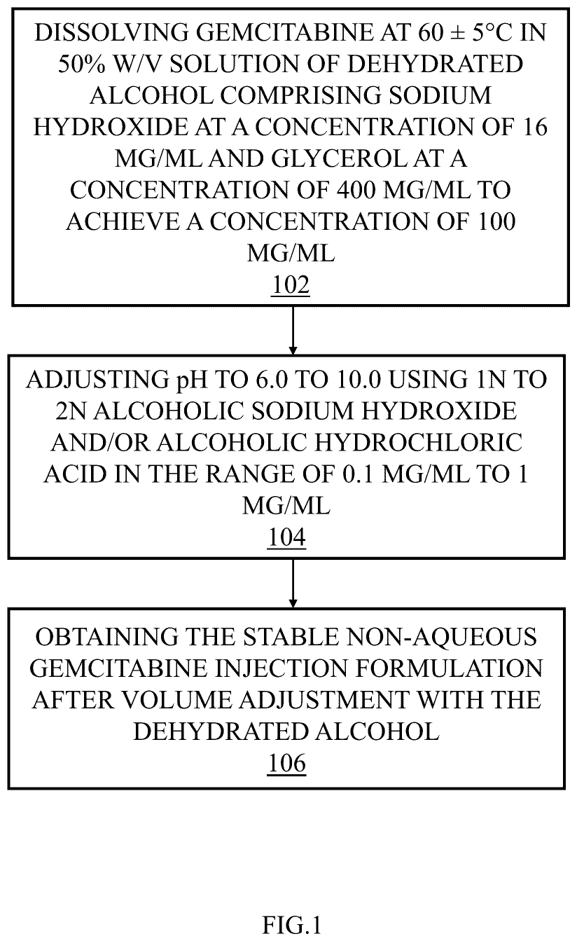

FIGS. 1A-B illustrate a method of preparing a stable non-aqueous Gemcitabine injection formulation according to various embodiments of the present invention.

At step 102, the method includes dissolving Gemcitabine at 60±5° C. in 50% w/v solution of dehydrated alcohol comprising sodium hydroxide at a concentration of 16 mg/mL and glycerol at a concentration of 400 mg/mL to achieve a concentration of 100 mg/mL.

At step 104, the method includes adjusting pH to 6.0 to 10.0 using 1N to 2N alcoholic sodium hydroxide and/or alcoholic hydrochloric acid in the range of 0.1 mg/mL to 1 mg/mL.

At step 106, the method includes obtaining the stable non-aqueous Gemcitabine injection formulation after volume adjustment with the dehydrated alcohol.

In an embodiment, the dehydrated alcohol is ethanol. The method includes preparing a diluted infusion of the formulation at concentrations of 0.1 mg/mL and 10 mg/mL with an infusion medium, which remains physically and chemically stable for 24 hours at room temperature. The infusion medium is a saline solution comprising 0.9% sodium chloride injection.

The present formulation simplifies the composition by utilizing glycerol as the sole solubilizer. Glycerol, an FDA-approved and widely used safe adjuvant in drug delivery systems, ensures safety and regulatory compliance. Additionally, the ready-to-use liquid form eliminates the need for reconstitution, simplifying administration and eradicating risks such as contamination, medication errors, and additional workload for healthcare personnel. The inclusion of sodium hydroxide and hydrochloric acid to adjust the pH further enhances the physical and chemical stability of the drug product, making it a stable, safe, and efficient alternative to existing formulations. The improved formulation of the present invention provides a stable drug product with stable infusions.

### Example 1

Preparation of Gemcitabine Injection (100 mg/mL):

Approximately 75 mL of a dehydrated alcohol was taken in a bottle. To it 2.4 μm of sodium hydroxide was added and stirred to obtain a clear first solution. To the first solution, 60 μm of glycerol was added and stirred to obtain a clear second solution. The temperature of second solution was maintained at 60±5° C. To it 17.129 μm of Gemcitabine hydrochloride API was added and stirred to obtain a clear third solution. The pH of third solution was adjusted using 1 N Hydrochloric acid to a range of 6.0 to 10.0. The volume of pH adjusted solution was made up to 150 mL by using the dehydrated alcohol. The injectable gemcitabine formulation was filtered through a 0.2 μm filter and the filtered solution is filled into each vial, then stoppered with rubber stoppers and sealed with flip-off seals.

TABLE 1
Stability data for Gemcitabine Injection (100 mg/mL):
Assay and impurity profile under long-term and
accelerated storage conditions for 6 months.

| | | | Long Term | Accelerated |
|---|---|---|---|---|
| | | | Condition | Condition |
| | Specifi- | | (25° C./ | (40° C./ |
| | cation | | 60% RH) | 75% RH) |
| Parameters | Limits | Initial | 6 Month | 6 Month |
| Assay | 90%-110% | 103.5 | 102.9 | 102.8 |
| Cytosine Imp | NMT 0.15% | ND | 0.001 | 0.001 |
| Gemcitabine | NMT 0.15%. | 0.003 | 0.001 | 0.006 |
| alpha anomer | | | | |
| 2- Deoxy 2,2 | NMT 0.50% | 0.062 | 0.102 | 0.116 |
| difluororudine | | | | |
| Highest | NMT 0.10% | 0.003 | 0.008 | 0.092 |
| Unknown | | | | |
| Total Imp | NMT 1.0% | 0.075 | 0.12 | 0.277 |

The table 1 presents stability data of the gemcitabine formulation stored under long-term (25° C./60% RH) and accelerated (40° C./75% RH) conditions over six months. The assay results indicate that the active ingredient concentration remains within the acceptable range of 90%-110%, consistent across both conditions. Impurity levels gradually increase over time, particularly under accelerated conditions. Despite these increases, all impurities, including the highest unknown impurity, remain within their specified limits. The total impurities rise from an initial 0.075% to 0.277% under accelerated conditions after six months, still well below the 1.0% limit. Overall, the gemcitabine formulation maintains its stability within acceptable ranges across both storage conditions.

### Example 2

Preparation of Gemcitabine Injection (100 mg/mL):

Approximately 250 mL of a dehydrated alcohol was taken in a bottle. To it 8.0 μm of sodium hydroxide was added and stirred to obtain a clear first solution. To the first solution, 200 μm of glycerol was added and stirred to obtain a clear second solution. The temperature of second solution was maintained at 60±5° C. To it 57.096 μm of Gemcitabine hydrochloride API was added and stirred to obtain a clear third solution. The pH of third solution was adjusted using 1 N Hydrochloric acid to a range of 6.0 to 9.0. The volume of pH adjusted solution was made up to 500 mL by using the dehydrated alcohol. The injectable gemcitabine formulation was filtered through a 0.2 μm PTFE filter, and the filtered solution was filled into each vial, then stoppered with rubber stoppers and sealed with flip-off seals.

TABLE 2
Stability data for Gemcitabine Injection (100 mg/mL):
Assay and impurity profile under long-term and
accelerated storage conditions for 3 months.

| | | | Long Term | Accelerated |
|---|---|---|---|---|
| | | | Condition | Condition |
| | Specifi- | | (25° C./ | (40° C./ |
| | cation | | 60% RH) | 75% RH) |
| Parameters | Limits | Initial | 3 Month | 3 Month |
| Assay | 90%-110% | 97.5 | 98.6 | 99.4 |
| Cytosine Imp | NMT 0.15% | ND | 0.001 | 0.002 |
| Gemcitabine | NMT 0.15%. | ND | ND | ND |
| alpha anomer | | | | |
| 2- Deoxy 2,2 | NMT 0.50% | 0.028 | 0.030 | 0.034 |
| difluororudine | | | | |
| Highest | NMT 0.10% | 0.03 | 0.018 | 0.048 |
| Unknown | | | | |
| Total Imp | NMT 1.0% | 0.095 | 0.10 | 0.184 |

The table 2 presents stability data of the gemcitabine formulation stored under long-term (25° C./60% RH) and accelerated (40° C./75% RH) conditions over three months. The assay results indicate that the active ingredient concentration remains within the acceptable range of 90%-110%, consistent across both conditions. Impurity levels gradually increase over time, particularly under accelerated conditions. Despite these increases, all impurities, including the highest unknown impurity, remain within their specified limits. The total impurities rise from an initial 0.095% to 0.184% under accelerated conditions after three months, still well below the 1.0% limit. Overall, the gemcitabine formulation maintains its stability within acceptable ranges across both storage conditions.

While the present invention has been described in terms of particular embodiments and applications, in both summarized and detailed forms, it is not intended that these descriptions in any way limit its scope to any such embodiments and applications. It will be understood that many substitutions, changes and variations in the described embodiments, applications and details of the method and system illustrated herein and of their operation can be made by those skilled in the art without departing from the spirit of this invention.

What is claimed is:

1. A stable non-aqueous Gemcitabine injection formulation comprising:
Gemcitabine at a concentration of 100 mg/mL;
glycerol at a concentration of 400 mg/mL as a solvent;
sodium hydroxide at a concentration of 16 mg/mL as a buffering agent; and
dehydrated alcohol as a vehicle at a concentration of 50% w/v, wherein the formulation is stable at a pH range of 6.0 to 10.0, wherein the pH of the formulation is adjusted using 1N to 2N alcoholic sodium hydroxide and/or alcoholic hydrochloric acid in the range of 0.1 mg/mL to 1 mg/mL.

2. The formulation of claim 1, wherein the dehydrated alcohol is ethanol.

3. The formulation of claim 1, wherein the formulation when diluted to concentrations of 0.1 mg/mL and 10 mg/mL with an infusion medium, results in an infusion that remains physically and chemically stable for a period of 24 hours at room temperature.

4. The formulation of claim 3, wherein the infusion medium is a normal saline solution comprising 0.9% sodium chloride.

5. A method for preparing a stable non-aqueous Gemcitabine injection formulation, wherein the method comprises, (i) dissolving Gemcitabine at 60±5° C. in 50% w/v solution of dehydrated alcohol comprising sodium hydroxide at a concentration of 16 mg/mL and glycerol at a concentration of 400 mg/mL to achieve a concentration of 100 mg/mL; adjusting pH to 6.0 to 10.0 using 1N to 2N alcoholic sodium hydroxide and/or alcoholic hydrochloric acid in the range of 0.1 mg/mL to 1 mg/mL; and (iii) obtaining the stable non-aqueous Gemcitabine injection formulation after volume adjustment with the dehydrated alcohol.

6. The method of claim 5, wherein the dehydrated alcohol is ethanol.

7. The method of claim 5, wherein the method comprises preparing a diluted infusion of the formulation at concentrations of 0.1 mg/mL and 10 mg/mL with an infusion medium, which remains physically and chemically stable for 24 hours at room temperature.

8. The method of claim 7, wherein the infusion medium is a normal saline solution comprising 0.9% sodium chloride.
